(19) United States
(12) Patent Application Publication
(10) Pub. No.: US 2022/0395442 A1
(43) Pub. Date: Dec. 15, 2022

(54) Methods of Modifying Biofilm

(71) Applicants: Colgate-Palmolive Company, New York, NY (US); Regents of the University of Michigan, Ann Arbor, MI (US)

(72) Inventors: Carlo DAEP, Brooklyn, NY (US); Deon HINES, Piscataway, NJ (US); Harsh Mahendra TRIVEDI, Hillsborough, NJ (US); James MASTERS, Ringoes, NJ (US); LaTonya KILPATRICK-LIVERMAN, Princeton, NJ (US); Luciana RINAUDI MARRON, Somerset, NJ (US); Lynette ZAIDEL, Cranford, NJ (US); Alexander H. RICKARD, Ann Arbor, MI (US); Derek S. SAMARIAN, Ann Arbor, MI (US); Rachel GICQUELAIS, Ann Arbor, MI (US); Gregory KRUSE, Ann Arbor, MI (US)

(73) Assignee: Colgate-Palmolive Company, New York, NY (US)

(21) Appl. No.: 17/774,971

(22) PCT Filed: Oct. 29, 2020
(86) PCT No.: PCT/US2020/070717

§ 371 Date: May 6, 2022

Related U.S. Application Data
(60) Provisional application No. 62/932,985, filed on Nov. 8, 2019.

(51) Int. Cl. A61K 8/44; A61K 8/49; A61K 8/27; A61K 8/19; A61Q 17/00; A61Q 11/00

(52) U.S. Cl. CPC A61K 8/44; A61K 8/4946; A61K 8/27; A61K 8/19; A61Q 17/005; A61Q 11/00; A61K 2800/92; A61K 2800/28

(57) ABSTRACT

Described herein are methods of treating biofilm (e.g. modifying surface topography, reducing thickness or reducing biovolume) and compositions and apparatus for use in carrying out such methods.

### BACKGROUND

Biofilms are defined as sessile communities characterized by cells that are reversibly attached to a surface or to each other, embedded in a matrix of extracellular polymeric substances. A biofilm community can be formed by a single kind of microorganism, but in nature, biofilms almost always consist of mixtures of many species of bacteria. For example, over five hundred bacterial species have been identified in typical dental plaque biofilms.

Microorganisms present in a biofilm can have significantly different properties from free-floating microorganisms of the same species. This is because the polymeric extracellular matrix acts to protect the microorganisms from the surrounding environment, which allows the microorganisms to cooperate and interact in various ways which are not exhibited by free-floating microorganisms. These complex communities of microorganisms present a unique challenge in that they are often resistant to classical means of antimicrobial control. Bacteria living in a biofilm exhibit increased resistance to antibiotics because the dense extracellular matrix and the outer layer of cells protect the interior of the biofilm from the effects of the antibiotics. Therefore, known antimicrobial agents will not have the same effect on bacteria present in a biofilm.

Despite advances in oral care therapeutics, there remains a need for new approaches to addressing the problems presented these oral biofilms. For example, reducing the ability of the biofilms to attach, or remain attached, to an oral cavity surface; compromising the structural integrity of the biofilms; and/or making the biofilms more susceptible to penetration by antimicrobial agents.

Embodiments of the present invention are designed to address these, and other, needs.

### BRIEF SUMMARY

In some embodiments, the present invention provides methods for modifying or manipulating the surface topography of an oral biofilm comprising administering an oral care composition comprising an effective amount of a basic amino acid to the oral cavity of a subject in need thereof.

Further embodiments of the present invention provide methods for reducing the thickness of an oral biofilm comprising administering an oral care composition comprising an effective amount of a basic amino acid to the oral cavity of a subject in need thereof.

Yet other embodiments of the present invention provide methods of decreasing the biovolume of an oral biofilm comprising administering an oral care composition comprising an effective amount of a basic amino acid to the oral cavity of a subject in need thereof.

In some embodiments, the roughness, thickness and/or biovolume are measured using a system comprising a frame having a first side, a second side, and an aperture extending from the first side to the second side; wherein a first cover is attached to the first side and covers the aperture; a second cover is attached to the second side and covers the aperture; and wherein an electromagnetic imaging device is configured to image the one or more biological samples through the first cover. In some embodiments, the biological sample comprises a biofilm (e.g. an oral biofilm).

In some embodiments, the frame may have a plurality of apertures which extend from the first side to the second side.

In other embodiments, the frame has a second aperture extending from the first side to the second side, a third cover attached to the first side and covering the second aperture, and a fourth cover attached to the second side and covering the second aperture.

In some embodiments, the system further includes a pH probe extending through the frame into the aperture.

In certain embodiments, the first cover is transparent.

In further embodiments, the second cover is transparent.

Some embodiments provide a frame having third and fourth sides, the third side having a first longitudinal passageway and the fourth side having a second longitudinal passageway, the first and second longitudinal passageways extending through the frame to the aperture.

In other embodiments, a first tube fitting is installed in the first longitudinal passageway and a second tube fitting is installed in the second longitudinal passageway, a first tube attached to the first tube fitting and a second tube attached to the second tube fitting.

In some embodiments, the second cover has a depression, the depression fluidly coupled with the aperture. In still further embodiments, a sample is located in the depression of the second cover.

In another embodiment, the first cover and the second cover are attached with an adhesive comprising silicone.

In another embodiment, the system includes a stand having two depressions, the frame having two arms that engage the two depressions of the stand to maintain the frame in an upright orientation.

In another embodiment, a plurality of protrusions extend from the second side of the frame. In some embodiments, the second cover is positioned between two of the plurality of protrusions.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The features, and advantages of the invention will be apparent from the following more detailed description of certain embodiments of the invention and as illustrated in the accompanying drawings in which:

FIG. 1 depicts the impact of certain compositions on biofilm biovolume, thickness and roughness;

FIG. 2 depicts the impact of certain compositions on biofilm biovolume, thickness and roughness;

FIG. 3 depicts biofilms treated in accordance with certain embodiments of the present invention;

FIG. 4 depicts biofilms grown for twenty (20) hours and treated in accordance with certain embodiments of the present invention;

FIG. 5 depicts biofilms treated in accordance with certain embodiments of the present invention; and

FIG. 6 depicts biofilms grown for twenty (20) hours and treated in accordance with certain embodiments of the present invention.

### DETAILED DESCRIPTION

The following description of the preferred embodiment(s) is merely exemplary in nature and is in no way intended to limit the invention, its application, or uses. Accordingly, the invention expressly should not be limited to such exemplary embodiments illustrating some possible non-limiting combination of features that may exist alone or in other combinations of features; the scope of the invention being defined by the claims appended hereto.

The description of illustrative embodiments according to principles of the present invention is intended to be read in connection with the accompanying drawings, which are to be considered part of the entire written description.

As used throughout, ranges are used as shorthand for describing each and every value that is within the range. Any value within the range can be selected as the terminus of the range.

In addition, all references cited herein are hereby incorporated by reference in their entireties. In the event of a conflict in a definition in the present disclosure and that of a cited reference, the present disclosure controls.

In some embodiments, the present invention provides methods for modifying or manipulating the surface topography of an oral biofilm comprising administering an oral care composition comprising an effective amount of a basic amino acid to the oral cavity of a subject in need thereof. In some embodiments, the basic amino acid is selected from arginine, lysine and histidine. In some embodiments, the basic amino acid is in free or salt form. In some embodiments, the basic amino acid is arginine. In further embodiments, the arginine comprises L-arginine.

In some embodiments, the effective amount of amino acid is an amount required to modify the roughness of a portion of an oral biofilm. In other embodiments, the effective amount of amino acid is an amount required to increase the roughness of a portion of an oral biofilm. In some embodiments, the roughness of a portion of the biofilm is increased by at least about 50%, at least about 55%, at least about 60%, at least about 65%, at least about 70%, at least about 75%, at least about 80%, at least about 85%, at least about 90%, at least about 95%, at least about 100%, at least about 105%, at least about 110%, at least about 115%, at least about 120%, at least about 125%, at least about 130%, at least about 135%, at least about 140%, at least about 145%, at least about 150%, at least about 200%, at least about 250%, at least about 300%, at least about 350%, at least about 4000%, at least about 450%, at least about 500%, at least about 550%, at least about 600%, at least about 650%, or about 700%.

For avoidance of doubt, as discussed herein, an increase in roughness can be calculated with respect to the roughness of an untreated biofilm or the roughness of a biofilm post-treatment with a control composition.

As used herein, “surface roughness” refers to the microscopic structural texture of a biofilm surface.

In some embodiments, roughness is measured using an apparatus comprising: a frame having a first side, a second side, and an aperture extending from the first side to the second side; a first cover attached to the first side and covering the aperture; a second cover attached to the second side and covering the aperture, a well formed by the frame, the first cover, and the second cover; a first tube fluidly coupled to the well; and a second tube fluidly coupled to the well.

In other embodiments, roughness is measured with a skidded gage.

Surface roughness can be measured in terms of a number of parameters known in the art. In some embodiments, roughness is calculated using a parameter selected from: Arithmetic Average Roughness (Ra); Root Mean Square (RMS) roughness (Rq); Maximum Peak Height (Rp); Maximum Valley Depth (Rv); Mean Roughness Depth (Rz); Maximum Roughness Depth (Rt); and Maximum Surface Roughness (Rmax).

In some embodiments, surface roughness is measured in terms of average surface roughness (Ra). Ra is the arithmetic average height of roughness component irregularities from the mean line measured within the sampling length. Smaller Ra values indicate smoother surfaces. Surface roughness can be measured by any method known in the art for measuring Ra, such as surface profilometry, surface scanning methods, confocal microscopy, atomic force microscopy, and scanning electron microscopy. Surface roughness can be measured before or after at least one treatment session and prior to any subsequent substantial exposure to other agents, for instance, remineralizing solutions (including saliva), or test agents.

In some embodiments, Ra values can range from about 2500 nm to about 5 nm, from 2000 nm to about 110 nm, from about 1000 nm to about 40 nm, from about 750 nm to about 40 nm, about 250 nm to about 20 nm, from about 200 nm to about 60 nm, about 50 nm, about 40 nm or about 30 nm. In other embodiments, the average Ra is greater than about 250 nm.

In some embodiments, the oral care composition is administered to the oral cavity for from about 10 seconds to about 30 minutes, e.g. about 15 seconds to about 25 minutes, about 20 seconds to about 15 minutes, about 25 seconds to about 5 minutes, or about 30 seconds to about 2 minutes. In some embodiments, the oral care compositions may be administered in a form that is maintained in the oral cavity overnight.

In some embodiments, the oral care composition is in a form selected from a toothpaste; a gel; a mouthwash; a prophy; a spray; a lozenge; a tablet, a capsule; a strip; a patch; and a dissolvable film.

In some embodiments, the oral biofilm is a mixed-species biofilm. In some embodiments, modifying or manipulating the surface topography (e.g. roughness) of the oral biofilm compromises the structural integrity of the biofilm. In other embodiments, the methods described herein make the biofilm more susceptible to penetration by an active ingredient. In still further embodiments, the attachment strength of the biofilm is reduced. While in other embodiments, at least a portion of the biofilm is dislodged from an oral cavity surface.

Further embodiments provide methods further comprising the step of expectorating at least a portion of the oral care composition. In some embodiments, at least a portion of the biofilm is removed from the oral cavity.

Still further embodiments provide methods for reducing the thickness of an oral biofilm comprising administering an oral care composition comprising an effective amount of a basic amino acid to the oral cavity of a subject in need thereof. In some embodiments, the thickness of a portion of the biofilm is decreased by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%/0, at least about 95%.

Other embodiments provide methods for reducing the biovolume of an oral biofilm comprising administering an oral care composition comprising an effective amount of a basic amino acid to the oral cavity of a subject in need thereof. In some embodiments, the biovolume of the biofilm is decreased by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%.

In some embodiments, the methods described herein further provide an anti-caries benefit. While in other embodiments, the methods described herein reduce of the risk of developing caries. In further embodiments, the methods described herein treat caries. Yet other embodiments provide methods for reducing the risk of developing periodontitis.

Still further embodiments provide methods for inhibiting the growth of a biofilm on an inanimate surface comprising applying an effective amount of any one of the compositions described herein to the inanimate surface. Other embodiments provide methods for removing of a biofilm from an inanimate surface comprising applying an effective amount of any one of the compositions described herein to the inanimate surface. In other embodiments, the present invention provides methods for modifying the surface topography (e.g. surface roughness) of a biofilm on an inanimate surface comprising applying an effective amount of any one of the compositions described herein to the inanimate surface.

Yet other embodiments provide methods for: modifying the surface topography of a biofilm; reducing the biovolume of a biofilm; and/or reducing the thickness of a biofilm, comprising applying an effective amount of any one of the compositions described herein to the biofilm. In some embodiments, a composition comprising an effective amount of a basic amino acid is applied to the biofilm. In some embodiments, the biofilm is present on an inanimate surface. In some embodiments, any one of the compositions described herein is applied directly to the inanimate surface. In some embodiments, the inanimate surface is a hard surface. In some embodiments, the inanimate surface is selected from a dental appliance; a dental implant; an industrial piping system; lab equipment; a water distribution system: and a food processing system.

In some embodiments the oral care compositions comprise insoluble to soluble ratios of metals e.g. zinc oxide to zinc citrate in a ratio from 1.5:1 to 4.5:1, 1.5:1 to 4:1, 1.7:1 to 2.3:1, 1.9:1 to 2.1:1, or about 2:1. Also, the corresponding molar ratios based on these weight ratios can be used. In some embodiments, the total concentration of zinc salts in the composition is from 0.2 weight % to 5 weight %, or from 0.5 weight % to 2.5 weight % or from 0.8 weight % to 2 weight %, or about 1.5 weight %, based on the total weight of the composition. In some embodiments, the molar ratio of arginine to total zinc salts is from 0.05:1 to 10:1. In some embodiments, the composition comprises zinc oxide in an amount of from 0.5 weight % to 1.5 weight % and zinc citrate in an amount of from 0.25 weight % to 0.75 weight %, based on the total weight of the composition. In some embodiments, the composition may comprise zinc oxide in an amount of from 0.75 weight % to 1.25 weigh % and zinc citrate in an amount of from 0.4 weight % to 0.6 weight %, based on the total weight of the composition. In some embodiments, the composition comprises zinc oxide in an amount of about 1 weight % and zinc citrate in an amount of about 0.5 weight %, based on the total weight of the composition. In some embodiments, zinc oxide may be present in an amount of from 0.75 to 1.25 wt % (e.g., 1.0 wt. %) the zinc citrate is in an amount of from 0.25 to 1.0 wt % (e.g. 0.25 to 0.75 wt. %, or 0.5 wt. %) and based on the weight of the oral care composition. In some embodiments, the zinc citrate is about 0.5 wt %. In some embodiments, the zinc oxide is about 1.0 wt %.

In some embodiments the ZnO particles may have an average particle size of from 1 to 7 microns. In some embodiments, the ZnO particles have an average particle size of 5 microns or less. In some embodiments, suitable zinc oxide particles for oral care compositions have, for example, a particle size distribution of 3 to 4 microns, or alternatively, a particle size distribution of 5 to 7 microns, alternatively, a particle size distribution of 3 to 5 microns, alternatively, a particle size distribution of 2 to 5 microns, or alternatively, a particle size distribution of 2 to 4 microns. Zinc oxide may have a particle size which is a median particle size. Suitable particles may have, for example, a median particle size of 8 microns or less, alternatively, a median particle size of 3 to 4 microns, alternatively, a median particle size of 5 to 7 microns, alternatively, a median particle size of 3 to 5 microns, alternatively, a median particle size of 2 to 5 microns, or alternatively, a median particle size of 2 to 4 microns. In another aspect, that particle size is an average (mean) particle size. In an embodiment, the mean particle comprises at least 5%, at least 10%, at least 15%, at least 20%, at least 25%, at least 30%, at least 35%, or at least 40% of the total metal oxide particles in an oral care composition of the invention. The particle may be present in an amount of up to 5% by weight, based on the total weight of the oral care composition, for example in an amount of from 0.5 to 5% by weight, preferably of up to 2% by weight, more preferably from 0.5 to 2% by weight, more preferably from 1 to 2% by weight, or in some embodiment from 2.5 to 4.5% by weight, being based on the total weight of the oral care composition. In some embodiments, the source of zinc oxide particles and/or the form they may be incorporated into the oral care composition in is selected from one or more of a powder, a nanoparticle solution or suspension, or encapsulated in a polymer or bead. Zinc oxide particles may be selected to achieve occlusion of dentin particles. Particle size distribution may be measured using a Malvern Particle Size Analyzer, Model Mastersizer 2000 (or comparable model) (Malvern Instruments, Inc., Southborough, Mass.), wherein a helium-neon gas laser beam is projected through a transparent cell which contains silica, such as, for example, silica hydrogel particles suspended in an aqueous solution. Light rays which strike the particles are scattered through angles which are inversely proportional to the particle size. The photodetector arrant measures the quantity of light at several predetermined angles. Electrical signals proportional to the measured light flux values are then processed by a microcomputer system, against a scatter pattern predicted from theoretical particles as defined by the refractive indices of the sample and aqueous dispersant to determine the particle size distribution of the metal oxide. It will be understood that other methods of measuring particle size are known in the art, and based on the disclosure set forth herein, the skilled artisan will understand how to calculate median particle size, mean particle size, and/or particle size distribution of metal oxide particles.

Oral care compositions comprise arginine or a salt thereof. In some embodiments, the arginine is L-arginine or a salt thereof. Suitable salts include salts known in the art to be pharmaceutically acceptable salts are generally considered to be physiologically acceptable in the amounts and concentrations provided. Physiologically acceptable salts include those derived from pharmaceutically acceptable inorganic or organic acids or bases, for example acid addition salts formed by acids which form a physiological acceptable anion, e.g., hydrochloride or bromide salt, and base addition salts formed by bases which form a physiologically acceptable cation, for example those derived from alkali metals such as potassium and sodium or alkaline earth metals such as calcium and magnesium. Physiologically acceptable salts may be obtained using standard procedures known in the art, for example, by reacting a sufficiently basic compound such as an amine with a suitable acid affording a physiologically acceptable anion. In some embodiments, the arginine in partially or wholly in salt form such as arginine phosphate, arginine hydrochloride or arginine bicarbonate. In some embodiments, the arginine is present in an amount corresponding to 0.1% to 15%, e.g., 0.1 wt % to 10 wt %, e.g., 0.1 to 5 wt %, e.g., 0.5 wt % to 3 wt % of the total composition weight, about e.g., 1%, 1.5%, 2%, 3%, 4%/o, 5%, or 8%, wherein the weight of the arginine is calculated as free form. In some embodiments the arginine is present in an amount corresponding to about 0.5 wt. % to about 20 wt. % of the total composition weight, about 0.5 wt. % to about 10 wt. % of the total composition weight, for example about 1.5 wt. %, about 3.75 wt. %, about 5 wt. %, or about 7.5 wt. % wherein the weight of the arginine is calculated as free form. In some embodiments, the arginine is present in an amount of from 0.5 weight % to 10 weight %, or from 0.5 weight % to 3 weight % or from 1 weight % to 2.85 weight %, or from 1.17 weight % to 2.25 weight %, based or from 1.4 weight % to 1.6 weight %, or from 0.75 weight % to 2.9 weight %, or from 1.3 weight % to 2 weight %, or about 1.5 weight %, based on the total weight of the composition. Typically, the arginine is present in an amount of up to 5% by weight, further optionally from 0.5 to 5% by weight, still further optionally from 2.5 to 4.5% by weight, based on the total weight of the oral care composition. In some embodiments, arginine is present in an amount from 0.1 wt. %-6.0 wt. %. (e.g., about 1.5 wt %) or from about 4.5 wt. %-8.5 wt. % (e.g., 5.0%) or from 3.5 wt. %-9 wt. % or 8.0 wt. %. In some embodiments, the arginine is present in a dentifrice, at for example about 0.5-2 wt. %, e.g., and about 0.8% in the case of a mouthwash.

One or more fluoride ion sources are optionally present in an amount providing a clinically efficacious amount of soluble fluoride ion to the oral care composition. A fluoride ion source is useful, for example, as an anti-caries agent. Any orally acceptable particulated fluoride ion source can be used, including stannous fluoride, sodium fluoride, potassium fluoride, potassium monofluorophosphate, sodium monofluorophosphate, ammonium monofluorophosphate, sodium fluorosilicate, ammonium fluorosilicate, indium fluoride, amine fluoride such as olaflur (N′-octadecyltrimethylendiamine-N,N,N′-tris(2-ethanol)-dihydrofluoride), ammonium fluoride, titanium fluoride, hexafluorosulfate, and combinations thereof. Fluoride where present may be present at levels of, e.g., about 25 to about 25,000 ppm, for example about 50 to about 5000 ppm, about 750 to about 2,000 ppm for a consumer toothpaste (e.g., 1000-1500 ppm, e.g., about 1000 ppm, e.g., about 1450 ppm), product. In some embodiments, fluoride is present from about 100 to about 1000, from about 200 to about 500, or about 250 ppm fluoride ion. 500 to 3000 ppm. In some embodiments, the fluoride source provides fluoride ion in an amount of from 50 to 25,000 ppm (e.g., 750-7000 ppm, e.g., 1000-5500 ppm, e.g., about 500 ppm, 1000 ppm, 1100 ppm, 2800 ppm, 5000 ppm, or 25000 ppm). In some embodiments, the fluoride source is stannous fluoride. In some embodiments, the fluoride source is stannous fluoride which provides fluoride in an amount from 750-7000 ppm (e.g., about 1000 ppm, 1100 ppm, 2800 ppm, 5000 ppm). In some embodiments, the fluoride source is stannous fluoride which provides fluoride in an amount of about 5000 ppm. In some embodiments, the fluoride source is sodium fluoride which provides fluoride in an amount from 750-2000 ppm (e.g., about 1450 ppm). In some embodiments, the fluoride source is selected from sodium fluoride and sodium monofluorophosphate and which provides fluoride in an amount from 1000 ppm-1500 ppm. In some embodiments, the fluoride source is sodium fluoride or sodium monofluorophosphate and which provides fluoride in an amount of about 1450 ppm. In some embodiments, stannous fluoride is the only fluoride source. In some embodiments, the fluoride source is stannous fluoride which provides fluoride in an amount from 750-7000 ppm (e.g., about 1000 ppm, 1100 ppm, 2800 ppm, 5000 ppm). In some embodiments, the fluoride source is stannous fluoride which provides fluoride in an amount of about 5000 ppm. Fluoride ion sources may be added to the compositions at a level of about 0.001 wt. % to about 10 wt. %, e.g., from about 0.003 wt. % to about 5 wt. %, 0.01 wt. % to about 1 wt., or about 0.05 wt. %. In some embodiment, the stannous fluoride is present in an amount of 0.1 wt. % to 2 wt. % (0.1 wt. %-0.6 wt. %) of the total composition weight. Fluoride ion sources may be added to the compositions at a level of about 0.001 wt. % to about 10 wt. %, e.g., from about 0.003 wt. % to about 5 wt. %, 0.01 wt. % to about 1 wt., or about 0.05 wt. %. However, it is to be understood that the weights of fluoride salts to provide the appropriate level of fluoride ion will obviously vary based on the weight of the counter ion in the salt, and one of skill in the art may readily determine such amounts. In some embodiment, the fluoride source is a fluoride salt present in an amount of 0.1 wt. % to 2 wt. % (0.1 wt %-0.6 wt. %) of the total composition weight (e.g., sodium fluoride (e.g., about 0.32 wt. %) or sodium monofluorophosphate). e.g., 0.3-0.4%, e.g., ca. 0.32% sodium fluoride

The oral care compositions described herein may also comprise one or more further agents such as those typically selected from the group consisting of: abrasives, an anti-plaque agent, a whitening agent, antibacterial agent, cleaning agent, a flavoring agent, a sweetening agent, adhesion agents, surfactants, foam modulators, pH modifying agents, humectants, mouth-feel agents, colorants, tartar control (anti-calculus) agent, polymers, saliva stimulating agent, nutrient, viscosity modifier, anti-sensitivity agent, antioxidant, and combinations thereof.

In some embodiments, the oral care compositions comprise one or more abrasive particulates such as those useful for example as a polishing agent. Any orally acceptable abrasive can be used, but type, fineness, (particle size) and amount of abrasive should be selected so that tooth enamel is not excessively abraded in normal use of the composition. Examples of abrasive particulates may be used include abrasives such sodium bicarbonate, insoluble phosphates (such as orthophosphates, polymetaphosphates and pyrophosphates including dicalcium orthophosphate dihydrate, calcium pyrophosphate, tricalcium phosphate, calcium polymetaphosphate and insoluble sodium polymetaphosphate), calcium phosphate (e.g., dicalcium phosphate dihydrate), calcium sulfate, natural calcium carbonate (CC), precipitated calcium carbonate (PCC), silica (e.g., hydrated silica or silica gels or in the form of precipitated silica or as admixed with alumina), iron oxide, aluminium oxide, aluminum silicate, calcined alumina, bentonite, other siliceous materials, perlite, plastic particles, e.g., polyethylene, and combinations thereof. The natural calcium carbonate abrasive of is typically a finely ground limestone which may optionally be refined or partially refined to remove impurities. The material preferably has an average particle size of less than 10 microns, e.g., 3-7 microns, e.g. about 5.5 microns. For example, a small particle silica may have an average particle size (D50) of 2.5-4.5 microns. Because natural calcium carbonate may contain a high proportion of relatively large particles of not carefully controlled, which may unacceptably increase the abrasivity, preferably no more than 0.01%, preferably no more than 0.004%) by weight of particles would not pass through a 325 mesh. The material has strong crystal structure, and is thus much harder and more abrasive than precipitated calcium carbonate. The tap density for the natural calcium carbonate is for example between 1 and 1.5 g/cc, e.g., about 1.2 for example about 1.19 g/cc. There are different polymorphs of natural calcium carbonate, e.g., calcite, aragonite and vaterite, calcite being preferred for purposes of this invention. An example of a commercially available product suitable for use in the present invention includes Vicron® 25-11 FG from GMZ. Precipitated calcium carbonate has a different crystal structure from natural calcium carbonate. It is generally more friable and more porous, thus having lower abrasivity and higher water absorption. For use in the present invention, the particles are small, e.g., having an average particle size of 1-5 microns, and e.g., no more than 0.1%, preferably no more than 0.05% by weight of particles which would not pass through a 325 mesh. The particles may for example have a D50 of 3-6 microns, for example 3.8-4.9, e.g., about 4.3; a D50 of 1-4 microns, e.g. 2.2-2.6 microns, e.g., about 2.4 microns, and a D10 of 1-2 microns, e.g., 1.2-1.4, e.g. about 1.3 microns. The particles have relatively high water absorption, e.g., at least 25 g/100 g, e.g. 30-70 g/100 g. Examples of commercially available products suitable for use include, for example, Carbolag® 15 Plus from Lagos Industria Quimica. In some embodiments, additional calcium-containing abrasives, for example calcium phosphate abrasive, e.g., tricalcium phosphate, hydroxyapatite or dicalcium phosphate dihydrate or calcium pyrophosphate, and/or silica abrasives, sodium metaphosphate, potassium metaphosphate, aluminum silicate, calcined alumina, bentonite or other siliceous materials, or combinations thereof are used. Examples of silica abrasives include, but are not limited to, precipitated or hydrated silicas having a mean particle size of up to about 20 microns (such as Zeodent 105 and Zeodent 1 14 marketed by J.M. Huber Chemicals Division, Havre de Grace, Md. 21078); Sylodent 783 (marketed by Davison Chemical Division of W.R. Grace & Company); or Sorbosil AC 43 (from PQ Corporation). In some embodiments, an effective amount of a silica abrasive is about 10-30%, e.g. about 20%. In some embodiments, the acidic silica abrasive Sylodent is included at a concentration of about 2 to about 35% by weight; about 3 to about 20% by weight, about 3 to about 15% by weight, about 10 to about 15% by weight. For example, the acidic silica abrasive may be present in an amount selected from 2 wt. %, 3 wt. %, 4% wt. %, 5 wt. %, 6 wt. %, 7 wt. %, 8 wt./o, 9 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %. Sylodent 783 has a pH of 3.4-4.2 when measured as a 5% by weight slurry in water and silica material has an average particle size of less than 10 microns, e.g., 3-7 microns, e.g. about 5.5 microns. In some embodiments, the silica is synthetic amorphous silica, (e.g., 1%-28% by wt.) (e.g., 8%-25% by wt). In some embodiments, the silica abrasives are silica gels or precipitated amorphous silicas, e.g. silicas having an average particle size ranging from 2.5 microns to 12 microns. Some embodiments further comprise a small particle silica having a median particle size (d50) of 1-5 microns (e.g., 3-4 microns) (e.g., about 5 wt. % Sorbosil AC43 from PQ Corporation Warrington, United Kingdom). The composition may contain from 5 to 20 wt % small particle silica, or for example 10-15 wt %, or for example 5 wt %, 10 wt %, 15 wt % or 20 wt % small particle silica. In some embodiments, 20-30 wt % of the total silica in the composition is small particle silica (e.g., having a median particle size (d50) of 3-4 microns and wherein the small particle silica is about 5 wt. % of the oral care composition. In some embodiments, silica is used as a thickening agent, e.g., particle silica. In some embodiments, the composition comprises calcium carbonate, such as precipitated calcium carbonate high absorption (e.g., 20% to 30% by weight of the composition or, 25% precipitated calcium carbonate high absorption), or precipitated calcium carbonate—light (e.g., about 10% precipitated calcium carbonate—light) or about 10% natural calcium carbonate.

In some embodiments, the oral care compositions comprise a whitening agent, e.g., a selected from the group consisting of peroxides, metal chlorites, perborates, percarbonates, peroxyacids, hypochlorites, hydroxyapatite, and combinations thereof. Oral care compositions may comprise hydrogen peroxide or a hydrogen peroxide source, e.g., urea peroxide or a peroxide salt or complex (e.g., such as peroxyphosphate, peroxycarbonate, perborate, peroxysilicate, or persulphate salts; for example, calcium peroxyphosphate, sodium perborate, sodium carbonate peroxide, sodium peroxyphosphate, and potassium persulfate or hydrogen peroxide polymer complexes such as hydrogen peroxide-polyvinyl pyrrolidone polymer complexes.

In some embodiments, the oral care compositions comprise an effective amount of one or more antibacterial agents, for example comprising an antibacterial agent selected from halogenated diphenyl ether (e.g. triclosan), triclosan monophosphate, herbal extracts and essential oils (e.g., rosemary extract, tea extract, magnolia extract, thymol, menthol, eucalyptol, geraniol, carvacrol, citral, hinokitol, magonol, ursolic acid, ursic acid, morin, catechol, methyl salicylate, epigallocatechin gallate, epigallocatechin, gallic acid, miswak extract, sea-buckthorn extract), bisguanide antiseptics (e.g., chlorhexidine, alexidine or octenidine), quaternary ammonium compounds (e.g., cetylpyridinium chloride (CPC), benzalkonium chloride, tetradecylpyridinium chloride (TPC), N-tetradecyl-4-ethylpyridinium chloride (TDEPC)), phenolic antiseptics, hexetidine furanones, bacteriocins, ethyllauroyl arginate, arginine bicarbonate, a Camellia extract, a flavonoid, a flavan, halogenated diphenyl ether, creatine, sanguinarine, povidone iodine, delmopinol, salifluor, metal ions (e.g., zinc salts, stannous salts, copper salts, iron salts), propolis and oxygenating agents (e.g., hydrogen peroxide, buffered sodium peroxyborate or peroxycarbonate), phthalic acid and its salts, monoperthalic acid and its salts and esters, ascorbyl stearate, oleoyl sarcosine, alkyl sulfate, dioctyl sulfosuccinate, salicylanilide, domiphen bromide, delmopinol, octapinol and other piperidino derivatives, nisin preparations, chlorite salts; parabens such as methylparaben or propylparaben and mixtures of any of the foregoing. One or more additional antibacterial or preservative agents may optionally be present in the composition in a total amount of from about 0.01 wt. % to about 0.5 wt. %, optionally about 0.05 wt. % to about 0.1 wt. % or about 0.3% by total weight of the composition.

In some embodiments, the oral care compositions may comprise at least one bicarbonate salt useful for example to impart a “clean feel” to teeth and gums due to effervescence and release of carbon dioxide. Any orally acceptable bicarbonate can be used, including without limitation, alkali metal bicarbonates such as sodium and potassium bicarbonates, ammonium bicarbonate and the like. The one or more additional bicarbonate salts are optionally present in a total amount of about 0.1 wt. % to about 50 wt. %, for example about 1 wt. % to 20 wt. %, by total weight of the composition.

In some embodiments, the oral care compositions also comprise at least one flavorant, useful for example to enhance taste of the composition. Any orally acceptable natural or synthetic flavorant can be used, including without limitation essential oils and various flavoring aldehydes, esters, alcohols, and similar materials, tea flavors, vanillin, sage, marjoram, parsley oil, spearmint oil, cinnamon oil, oil of wintergreen, peppermint oil, clove oil, bay oil, anise oil, eucalyptus oil, citrus oils, fruit oils, sassafras and essences including those derived from lemon, orange, lime, grapefruit, apricot, banana, grape, apple, strawberry, cherry, pineapple, etc., bean- and nut-derived flavors such as coffee, cocoa, cola, peanut, almond, etc., adsorbed and encapsulated flavorants and the like. Also encompassed within flavorants herein are ingredients that provide fragrance and/or other sensory effect in the mouth, including cooling or wanning effects. Such ingredients illustratively include menthol, carvone, menthyl acetate, menthyl lactate, camphor, eucalyptus oil, eucalyptol, anethole, eugenol, cassia, oxanone, a-irisone, propenyl guaiethoi, thymol, linalool, benzaldehyde, cinnamaldehyde, N-ethyl-p-menthan-3-carboxamine, N,2,3-trimethyl-2-isopropylbutanamide, 3-(1-menthoxy)-propane-1,2-diol, cinnamaldehyde glycerol acetal (CGA), menthone glycerol acetal (MGA) and the like. One or more flavorants are optionally present in a total amount of from about 0.01 wt. % to about 5 wt. %, for example, from about 0.03 wt. % to about 2.5 wt. %, optionally about 0.05 wt. % to about 1.5 wt. %, further optionally about 0.1 wt. % to about 0.3 wt. % and in some embodiments in various embodiments from about 0.01 wt. % to about 1 wt. %, from about 0.05 to about 2%, from about 0.1% to about 2.5%, and from about 0.1 to about 0.5% by total weight of the composition.

In some embodiments, the oral care compositions comprise at least one sweetener, useful for example to enhance taste of the composition. Sweetening agents among those useful herein include dextrose, polydextrose, sucrose, maltose, dextrin, dried invert sugar, mannose, xylose, ribose, fructose, levulose, galactose, corn syrup, partially hydrolyzed starch, hydrogenated starch hydrolysate, ethanol, sorbitol, mannitol, xylitol, maltitol, isomalt, aspartame, neotame, saccharin and salts thereof (e.g. sodium saccharin), sucralose, dipeptide-based intense sweeteners, cyclamates, dihydrochalcones, glycerine, propylene glycol, polyethylene glycols, Poloxomer polymers such as POLOXOMER 407, PLURONIC F108, (both available from BASF Corporation), alkyl polyglycoside (APG), polysorbate, PEG40, castor oil, menthol, and mixtures thereof. One or more sweeteners are optionally present in a total amount depending strongly on the particular sweetener(s) selected, but typically 0.005 wt. % to 5 wt. %, by total weight of the composition, optionally 0.005 wt. % to 0.2 wt. %, further optionally 0.05 wt. % to 0.1 wt. % by total weight of the composition.

In some embodiments, the oral care compositions further comprises an agent that interferes with or prevents bacterial attachment, e.g., ethyl lauroyl arginiate (ELA), solbrol or chitosan, as well as plaque dispersing agents such as enzymes (papain, glucoamylase, etc.).

In some embodiments, the oral care compositions also comprise at least one surfactant. Any orally acceptable surfactant, most of which are anionic, cationic, zwitterionic, nonionic or amphoteric, and mixtures thereof, can be used. Examples of suitable surfactants include water-soluble salts of higher fatty acid monoglyceride monosulfates, such as the sodium salt of monosulfated monoglyceride of hydrogenated coconut oil fatty acids; higher alkyl sulfates such as sodium lauryl sulfate, sodium coconut monoglyceride sulfonate, sodium lauryl sarcosinate, sodium lauryl isoethionate, sodium laureth carboxylate and sodium dodecyl benzenesulfonate; alkyl aryl sulfonates such as sodium dodecyl benzene sulfonate; higher alkyl sulfoacetates, such as sodium lauryl sulfoacetate; higher fatty acid esters of 1,2-dihydroxypropane sulfonate; and the substantially saturated higher aliphatic acyl amides of lower aliphatic amino carboxylic compounds, such as those having 12-16 carbons in the fatty acid, alkyl or acyl radicals; and the like. Examples of amides include N-lauryl sarcosine, and the sodium, potassium and ethanolamine salts of N-lauryl, N-myristoyl, or N-palmitoyl sarcosine. Examples of cationic surfactants include derivatives of aliphatic quaternary ammonium compounds having one long alkyl chain containing 8 to 18 carbon atoms such as lauryl trimethylammonium chloride, cetyl pyridinium chloride, cetyl trimethyl ammonium bromide, di-isobutylphenoxyethyldimethylbenzylammonium chloride, coconut alkyltrimethylammonium nitrite, cetyl pyridinium fluoride, and mixtures thereof. Suitable nonionic surfactants include without limitation, poloxamers, polyoxyethylene sorbitan esters, fatty alcohol ethoxylates, alkylphenol ethoxylates, tertiary amine oxides, tertiary phosphine oxides, di alkyl sulfoxides and the like. Others include, for example, non-anionic polyoxyethylene surfactants, such as Polyoxamer 407, Steareth 30, Polysorbate 20, and castor oil; and amphoteric surfactants such as derivatives of aliphatic secondary and tertiary amines having an anionic group such as carboxylate, sulfate, sulfonate, phosphate or phosphonate such as cocamidopropyl betaine (tegobaine), and cocamidopropyl betaine lauryl glucoside; condensation products of ethylene oxide with various hydrogen containing compounds that are reactive therewith and have long hydrocarbon chains (e.g., aliphatic chains of from 12 to 20 carbon atoms), which condensation products (ethoxamers) contain hydrophilic polyoxyethylene moieties, such as condensation products of poly (ethylene oxide) with fatty acids, fatty, alcohols, fatty amides and other fatty moieties, and with propylene oxide and polypropylene oxides. In some embodiments, the oral composition includes a surfactant system that is sodium laurel sulfate (SLS) and cocamidopropyl betaine. One or more surfactants are optionally present in a total amount of about 0.01 wt. % to about 10 wt. %, for example, from about 0.05 wt. % to about 5 wt. %, or from about 0.1 wt. % to about 2 wt. %, e.g. 1.5% wt. by total weight of the composition. In some embodiments, the oral composition include an anionic surfactant, e.g., a surfactant selected from sodium lauryl sulfate, sodium ether lauryl sulfate, and mixtures thereof, e.g. in an amount of from about 0.3% to about 4.5% by weight, e.g. 1-2% sodium lauryl sulfate (SLS); and/or a zwitterionic surfactant, for example a betaine surfactant, for example cocamidopropylbetaine, e.g. in an amount of from about 0.1% to about 4.5% by weight, e.g. 0.5-2° % cocamidopropylbetaine. Some embodiments comprise a nonionic surfactant in an amount of from 0.5-5%, e.g., 1-2%, selected from poloxamers (e.g., poloxamer 407), polysorbates (e.g., polysorbate 20), polyoxyl hydrogenated castor oil (e.g., polyoxyl 40 hydrogenated castor oil), and mixtures thereof. In some embodiments, the poloxamer nonionic surfactant has a polyoxypropylene molecular mass of from 3000 to 5000 g/mol and a polyoxyethylene content of from 60 to 80 mol %, e.g., the poloxamer nonionic surfactant comprises poloxamer 407. Any of the preceding compositions may further comprise sorbitol, wherein the sorbitol is in a total amount of 10-40% (e.g., about 23%).

In some embodiments, the oral care compositions comprise at least, one foam modulator, useful for example to increase amount, thickness or stability of foam generated by the composition upon agitation. Any orally acceptable foam modulator can be used, including without limitation, polyethylene glycols (PEGs), also known as polyoxyethylenes. High molecular weight PEGs are suitable, including those having an average molecular weight of 200,000 to 7,000,000, for example 500,000 to 5,000,000, or 1,000,000 to 2,500,000, One or more PEGs are optionally present in a total amount of about 0.1 wt. % to about 10 wt. %, for example from about 0.2 wt. % to about 5 wt. %, or from about 0.25 wt. % to about 2 wt. %, by total weight of the composition

In some embodiments, the oral care compositions comprise at least one pH modifying agent. Such agents include acidifying agents to lower pH, basifying agents to raise pH, and buffering agents to control pH within a desired range. For example, one or more compounds selected from acidifying, basifying and buffering agents can be included to provide a pH of 2 to 10, or in various illustrative embodiments, 2 to 8, 3 to 9, 4 to 8, 5 to 7, 6 to 10, 7 to 9, etc. Any orally acceptable pH modifying agent can be used, including without limitation, carboxylic, phosphoric and sulfonic acids, acid salts (e.g., monosodium citrate, disodium citrate, monosodium malate, etc.), alkali metal hydroxides such as sodium hydroxide, carbonates such as sodium carbonate, bicarbonates such as sodium bicarbonate, sesquicarbonates, borates, silicates, bisulfates, phosphates (e.g., monosodium phosphate, trisodium phosphate, monopotassium phosphate, dipotassium phosphate, tribasic sodium phosphate, sodium tripolyphosphate, phosphoric acid), imidazole, sodium phosphate buffer (e.g., sodium phosphate monobasic and disodium phosphate) citrates (e.g. citric acid, trisodium citrate dehydrate), pyrophosphates (sodium and potassium salts) and the like and combinations thereof. One or more pH modifying agents are optionally present in a total amount effective to maintain the composition in an orally acceptable pH range. Compositions may have a pH that is either acidic or basic, e.g., from pH 4 to pH 5.5 or from pH 8 to pH 10. In some embodiments, the amount of buffering agent is sufficient to provide a pH of about 5 to about 9, preferable about 6 to about 8, and more preferable about 7, when the composition is dissolved in water, a mouthrinse base, or a toothpaste base. Typical amounts of buffering agent are about 5% to about 35%, in one embodiment about 10% to about 30%), in another embodiment about 15% to about 25%, by weight of the total composition.

In some embodiments, the oral care compositions also comprise at least one humectant. Any orally acceptable humectant can be used, including without limitation, polyhydric alcohols such as glycerin, sorbitol (optionally as a 70 wt. % solution in water), propylene glycol, xylitol or low molecular weight polyethylene glycols (PEGs) and mixtures thereof. Most humectants also function as sweeteners. In some embodiments, compositions comprise 15% to 70% or 30% to 65% by weight humectant. Suitable humectants include edible polyhydric alcohols such as glycerin, sorbitol, xylitol, propylene glycol as well as other polyols and mixtures of these humectants. Mixtures of glycerine and sorbitol may be used in certain embodiments as the humectant component of the compositions herein. One or more humectants are optionally present in a total amount of from about 1 wt. % to about 70 wt. %, for example, from about 1 wt. % to about 50 wt. %, from about 2 wt. % to about 25 wt. %, or from about 5 wt. % to about 15 wt. %, by total weight of the composition. In some embodiments, humectants, such as glycerin are present in an amount that is at least 20%>, e.g., 20-40%, e.g., 25-35%.

Mouth-feel agents include materials imparting a desirable texture or other feeling during use of the composition. In some embodiments, the oral care compositions comprise at least one thickening agent, useful for example to impart a desired consistency and/or mouth feel to the composition. Any orally acceptable thickening agent can be used, including without limitation, carbomers, also known as carboxyvinyl polymers, carrageenans, also known as Irish moss and more particularly i-carrageenan (iota-carrageenan), cellulosic polymers such as hydroxyethyl cellulose, and water-soluble salts of cellulose ethers (e.g., sodium carboxymethyl cellulose and sodium carboxymethyl hydroxyethyl cellulose), carboxymethylcellulose (CMC) and salts thereof, e.g., CMC sodium, natural gums such as karaya, xanthan, gum arabic and tragacanthin, colloidal magnesium aluminum silicate, colloidal silica, starch, polyvinyl pyrrolidone, hydroxyethyl propyl cellulose, hydroxybutyl methyl cellulose, hydroxypropyl methyl cellulose, and hydroxyethyl cellulose and amorphous silicas, and the like. A preferred class of thickening or gelling agents includes a class of homopolymers of acrylic acid crosslinked with an alkyl ether of pentaerythritol or an alkyl ether of sucrose, or carbomers. Carbomers are commercially available from B.F. Goodrich as the Carbopol© series. Particularly preferred Carbopols include Carbopol 934, 940, 941, 956, 974P, and mixtures thereof. Silica thickeners such as DT 267 (from PPG Industries) may also be used. One or more thickening agents are optionally present in a total amount of from about 0.01 wt. % to 15 wt. %, for example from about 0.1 wt. % to about 10 wt. %, or from about 0.2 wt. % to about 5 wt. %, by total weight of the composition. Some embodiments comprise sodium carboxymethyl cellulose (e.g., from 0.5 wt. %-1.5 wt. %). In certain embodiments, thickening agents in an amount of about 0.5% to about 5.0% by weight of the total composition are used. Thickeners may be present in an amount of from 1 wt % to 15 wt %, from 3 wt % to 10 wt %, 4 wt % to 9 wt %, from 5 wt % to 8 wt ° %, for example 5 wt %, 6 wt %, 7 wt %, or 8 wt %.

In some embodiments, the oral care compositions comprise at least one colorant. Colorants herein include pigments, dyes, lakes and agents imparting a particular luster or reflectivity such as pearling agents. In various embodiments, colorants are operable to provide a white or light-colored coating on a dental surface, to act as an indicator of locations on a dental surface that have been effectively contacted by the composition, and/or to modify appearance, in particular color and/or opacity, of the composition to enhance attractiveness to the consumer. Any orally acceptable colorant can be used, including FD&C dyes and pigments, talc, mica, magnesium carbonate, calcium carbonate, magnesium silicate, magnesium aluminum silicate, silica, titanium dioxide, zinc oxide, red, yellow, brown and black iron oxides, ferric ammonium ferrocyanide, manganese violet, ultramarine, titaniated mica, bismuth oxychloride, and mixtures thereof. One or more colorants are optionally present in a total amount of about 0.001% to about 20%, for example about 0.01% to about 10% or about 0.1% to about 5% by total weight of the composition.

In some embodiments, the oral care composition further comprises an anti-calculus (tartar control) agent. Suitable anti-calculus agents include, but are not limited to: phosphates and polyphosphates, polyaminopropane sulfonic acid (AM PS), polyolefin sulfonates, polyolefin phosphates, diphosphonates such as azacycloalkane-2,2-diphosphonates (e.g., azacycloheptane-2,2-diphosphonic acid), N-methyl azacyclopentane-2,3-diphosphonic acid, ethane-1-hydroxy-1,1-diphosphonic acid (EHDP) and ethane-1-amino-1,1-diphosphonate, phosphonoalkane carboxylic acids and. Useful inorganic phosphate and polyphosphate salts include monobasic, dibasic and tribasic sodium phosphates. Soluble pyrophosphates are useful anticalculus agents. The pyrophosphate salts can be any of the alkali metal pyrophosphate salts. In certain embodiments, salts include tetra alkali metal pyrophosphate, dialkali metal diacid pyrophosphate, trialkali metal monoacid pyrophosphate and mixtures thereof, wherein the alkali metals are sodium or potassium. The pyrophosphates also contribute to preservation of the compositions by lowering water activity, tetrasodium pyrophosphate (TSPP), tetrapotassium pyrophosphate, sodium tripolyphosphate, tetrapolyphosphate, sodium trimetaphosphate, sodium hexametaphosphate and mixtures thereof. The salts are useful in both their hydrated and unhydrated forms. An effective amount of pyrophosphate salt useful in the present composition is generally enough to provide least 0.1 wt. % pyrophosphate ions, e.g., 0.1 to 3 wt. %, e.g., 0.1 to 2 wt. %, e.g., 0.1 to 1 wt. %, e.g., 0.2 to 0.5 wt. %.

Other useful tartar control agents include polymers and co-polymers. In some embodiments, the oral care compositions include one or more polymers, such as polyethylene glycols, polyvinyl methyl ether maleic acid copolymers, polysaccharides (e.g., cellulose derivatives, for example carboxymethyl cellulose, or polysaccharide gums, for example xanthan gum or carrageenan gum). Acidic polymers, for example polyacrylate gels, may be provided in the form of their free acids or partially or fully neutralized water-soluble alkali metal (e.g., potassium and sodium) or ammonium salts. Certain embodiments include 1:4 to 4:1 copolymers of maleic anhydride or acid with another polymerizable ethylenically unsaturated monomer, for example, methyl vinyl ether (methoxyethylene), having a molecular weight (M.W.) of about 30,000 to about 1,000,000, polyvinyl methyl ether/maleic anhydride (PVM/MA) copolymers such as GANTREZ® (e.g., GANTREZ® S-97 polymer). In some embodiments, the PVM/MA copolymer comprises a copolymer of methyl vinyl ether/maleic anhydride, wherein the anhydride is hydrolyzed following copolymerization to provide the corresponding acid. In some embodiments, PVM/MA copolymer has an average molecular weight (M.W.) of about 30,000 to about 1,000,000, e.g. about 300,000 to about 800,000, e.g., wherein the anionic polymer is about 1-5%, e.g., about 2%, of the weight of the composition. In some embodiments, the anti-calculus agent is present in the composition in an amount of from 0.2 weight % to 0.8 weight %; 0.3 weight % to 0.7 weight %; 0.4 weight % to 0.6 weight %; or about 0.5 weight %, based on the total weight of the composition. Copolymers are available for example as Gantrez AN 139(M.W. 500,000), AN 1 19 (M.W. 250,000) and S-97 Pharmaceutical Grade (M.W. 70,000), of GAF Chemicals Corporation. Other operative polymers include those such as the 1:1 copolymers of maleic anhydride with ethyl acrylate, hydroxyethyl methacrylate, N-vinyl-2-pyrollidone, or ethylene, the latter being available for example as Monsanto EMA No. 1 103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone. Suitable generally, are polymerized olefinically or ethyl enically unsaturated carboxylic acids containing an activated carbon-to-carbon olefinic double bond and at least one carboxyl group, that is, an acid containing an olefinic double bond which readily functions in polymerization because of its presence in the monomer molecule either in the alpha-beta position with respect to a carboxyl group or as part of a terminal methylene grouping. Illustrative of such acids are acrylic, methacrylic, ethacrylic, alpha-chloroacrylic, crotonic, beta-acryloxy propionic, sorbic, alpha-chlorsorbic, cinnamic, beta-styrylacrylic, muconic, itaconic, citraconic, mesaconic, glutaconic, aconitic, alpha-phenylacrylic, 2-benzyl acrylic, 2-cyclohexylacrylic, angelic, umbellic, fumaric, maleic acids and anhydrides. Other different olefinic monomers copolymerizable with such carboxylic monomers include vinylacetate, vinyl chloride, dimethyl maleate and the like. Copolymers contain sufficient carboxylic salt groups for water-solubility. A further class of polymeric agents includes a composition containing homopolymers of substituted acrylamides and/or homopolymers of unsaturated sulfonic acids and salts thereof, in particular where polymers are based on unsaturated sulfonic acids selected from acrylamidoalykane sulfonic acids such as 2-acrylamide 2 methylpropane sulfonic acid having a molecular weight of about 1,000 to about 2,000,000. Another useful class of polymeric agents includes polyamino acids, particularly those containing proportions of anionic surface-active amino acids such as aspartic acid, glutamic acid and phosphoserine.

In some embodiments, the oral care compositions comprise a saliva stimulating agent useful, for example, in amelioration of dry mouth. Any orally acceptable saliva stimulating agent can be used, including without limitation food acids such as citric, lactic, malic, succinic, ascorbic, adipic, fumaric and tartaric acids, and mixtures thereof. One or more saliva stimulating agents are optionally present in saliva stimulating effective total amount.

In some embodiments, the oral care compositions comprise a nutrient. Suitable nutrients may include vitamins, minerals, amino acids, and mixtures thereof.

In some embodiments, the oral care compositions comprise at least one viscosity modifier, useful for example to help inhibit settling or separation of ingredients or to promote re-dispersibility upon agitation of a liquid composition. Any orally acceptable viscosity modifier can be used, including without limitation, mineral oil, petrolatum, clays and organo-modified clays, silicas and the like. One or more viscosity modifiers are optionally present in a total amount of from about 0.01 wt. % to about 10 wt. %, for example, from about 0.1 wt. % to about 5 wt. %, by total weight of the composition.

In some embodiments, the oral care compositions comprise antisensitivity agents, e.g., potassium salts such as potassium nitrate, potassium bicarbonate, potassium chloride, potassium citrate, and potassium oxalate; capsaicin; eugenol; strontium salts; chloride salts and combinations thereof. Such agents may be added in effective amounts, e.g., from about 1 wt. % to about 20 wt. % by weight based on the total weight of the composition, depending on the agent chosen.

In some embodiments, the oral care compositions comprise an antioxidant. Any orally acceptable antioxidant can be used, including butylated hydroxy anisole (BHA), butylated hydroxytoluene (BHT), vitamin A, carotenoids, co-enzyme Q10, PQQ, Vitamin A, Vitamin C, vitamin E, anethole-dithiothione, flavonoids, polyphenols, ascorbic acid, herbal antioxidants, chlorophyll, melatonin, and mixtures thereof.

In some embodiments, the oral care compositions comprise of one or more alkali phosphate salts, e.g., sodium, potassium or calcium salts, e.g., selected from alkali dibasic phosphate and alkali pyrophosphate salts, e.g., alkali phosphate salts selected from sodium phosphate dibasic, potassium phosphate dibasic, dicalcium phosphate dihydrate, calcium pyrophosphate, tetrasodium pyrophosphate, tetrapotassium pyrophosphate, sodium tripolyphosphate, disodium hydrogenorthophoshpate, monosodium phosphate, pentapotassium triphosphate and mixtures of any of two or more of these, e.g., in an amount of 0.01-20%, e.g., 0.1-8%, e.g., e.g., 0.1 to 5%, e.g., 0.3 to 2%, e.g., 0.3 to 1%, e.g about 0.01%, about 0.1%, about 0.5%, about 1%, about 2%, about 5%, about 6%, by weight of the composition. In some embodiments, compositions comprise tetrapotassium pyrophosphate, disodium hydrogenorthophoshpate, monosodium phosphate, and pentapotassium triphosphate. In some embodiments, compositions comprise tetrasodium pyrophosphate from 0.1-1.0 wt % (e.g., about 0.5 wt %).

In some embodiments, the oral care compositions comprise a source of calcium and phosphate selected from (i) calcium-glass complexes, e.g., calcium sodium phosphosilicates, and (ii) calcium-protein complexes, e.g., casein phosphopeptide-amorphous calcium phosphate. Any of the preceding compositions further comprising a soluble calcium salt, e.g., selected from calcium sulfate, calcium chloride, calcium nitrate, calcium acetate, calcium lactate, and combinations thereof.

In some embodiments, the oral care compositions comprise an additional ingredient selected from: benzyl alcohol, methylisothizolinone (“MIT”), Sodium bicarbonate, sodium methyl cocoyl taurate (tauranol), lauryl alcohol, and polyphosphate. Some embodiments comprise benzyl alcohol that is present from 0.1-0.8 wt %, or 0.2 to 0.7 wt %, or from 0.3 to 0.6 wt %, or from 0.4 to 0.5 wt %, e.g. about 0.1 wt. %, about 0.2 wt. %, about 0.3 wt %, about 0.4 wt %, about 0.5 wt %, about 0.6 wt %, about 0.7 wt % or about 0.8 wt %.

In some embodiments, the oral care compositions comprise from 5%-40%, e.g., 10%-35%, e.g., about 15%, 25%, 30%, or 35% or more of water.

### EXAMPLES

### Example 1

Biofilms are grown at 37° C. for about twenty (20) hours and the flow rate of cell free saliva (CFS) supplemented with 0.1% sucrose is set to 6.43 μL/min. After growth, biofilms are treated with either 0.75 mL fifty percent (50%) toothpaste slurry in water over thirty (30) seconds or three (3) mL fifty percent (50%) toothpaste supernatant over two (2) minutes. A water treatment is used as a control. Following treatment, a washout (to remove particulate matter) is performed by running eleven (11) mL of water over the biofilm over one (1) hour. After the washout, the biofilms are stained with Syto9 and propidium iodide and imaged on a Leica SPE confocal using a 10× objective lens (NA=0.4).

Image rendering and image analysis using Imaris and Comstat2 showed a significant decrease in both biovolume and thickness between the biofilms treated with a Control (water) and biofilms treated with a toothpaste slurry comprising—inter alia—a calcium abrasive (Composition 1), or a toothpaste slurry comprising—inter alia—a calcium abrasive and 1.5% arginine (Composition 2). Specifically, compared to the Control, a reduction of 76% and 85% was seen in the biovolume of the biofilms treated with Composition 1 and Composition 2, respectively. Also compared to the Control, a drop of 80% and 90% in thickness was observed between biofilms treated with Composition 1 and Composition 2, respectively. The roughness of biofilms treated with Composition 1 and Composition 2 was 566% and 692% higher than the roughness of the control biofilms respectively. It was also observed that Composition 2 was more effective at removing biofilm compared to Composition 1. Live/Dead staining indicated no significant difference between slurries of Composition 1 and Composition 2. Treatment with either slurry increased the amount of red fluorescent signal compared to the Control-treated biofilm, indicating a significant amount of cell damage/death caused by these slurry treatments.

To avoid inter-run variation, biovolume, thickness, and roughness values from biofilms treated with Composition 1 and Composition 2 are normalized against control biofilms grown in parallel with the respective treated biofilms. Image rendering and image analysis using Imaris and Comstat2 showed a decrease in both biovolume and thickness between the Control-treated biofilms and biofilms treated with Composition 1 and Composition 2 supernatants. Specifically, compared to the Control, a reduction of 46% and 72% was seen in the biovolume of the biofilms treated with Composition 1 and Composition 2 supernatants, respectively.

Also compared to the Control, a drop of 60% and 81% in thickness was observed between biofilms treated with Composition 1 and Composition 2 supernatants, respectively. The roughness of biofilms treated with Composition 1 and Composition 2 supernatants was 103% and 157% higher than the roughness of the Control-treated biofilms, respectively. It was also observed that the Composition 2 supernatant was significantly more effective at reducing biofilm biovolume compared to Composition 1 supernatant. Again, Live/Dead staining indicated no significant difference between the Composition 1 and Composition 2 supernatants. Interestingly, both Composition 1 and Composition 2 raised the amount of red fluorescent signal from the biofilms, which suggests a significant amount of cell damage/death which is less likely to be linked to effects from particulate matter that is present in the slurries.

### Example 2

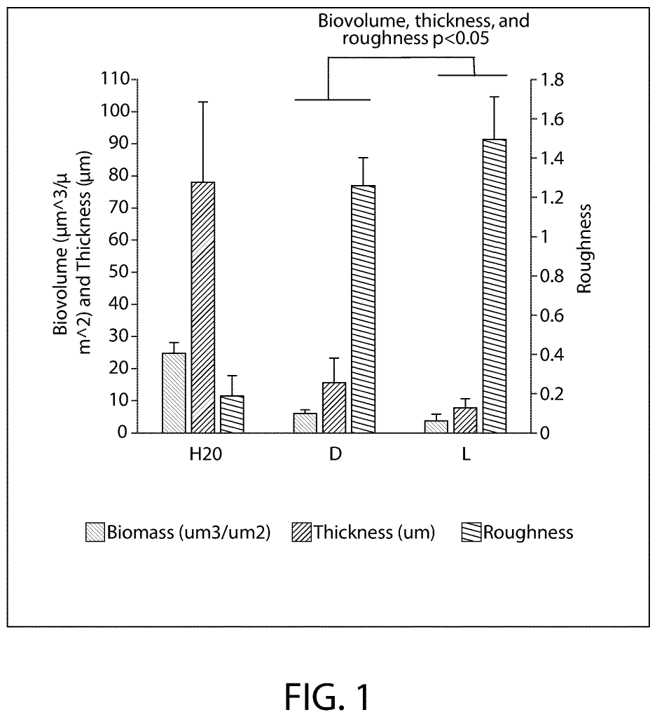

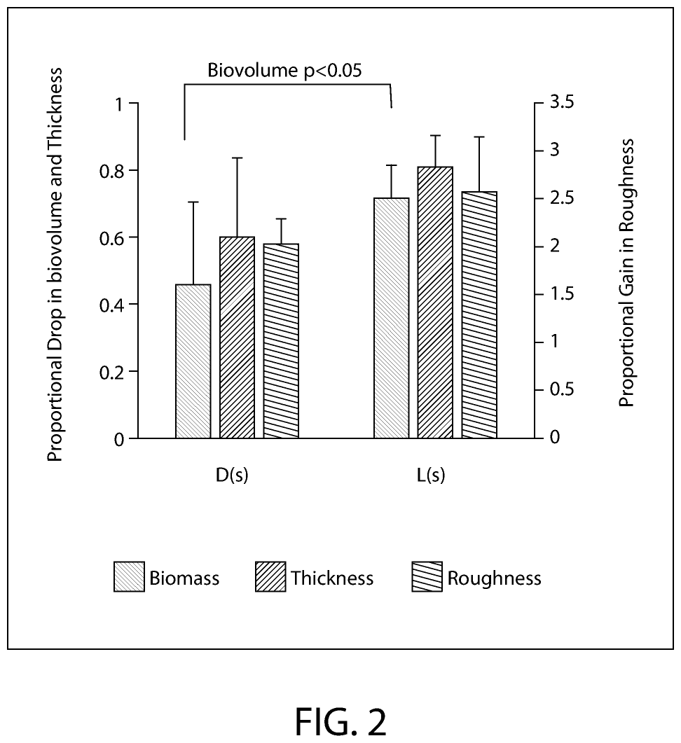

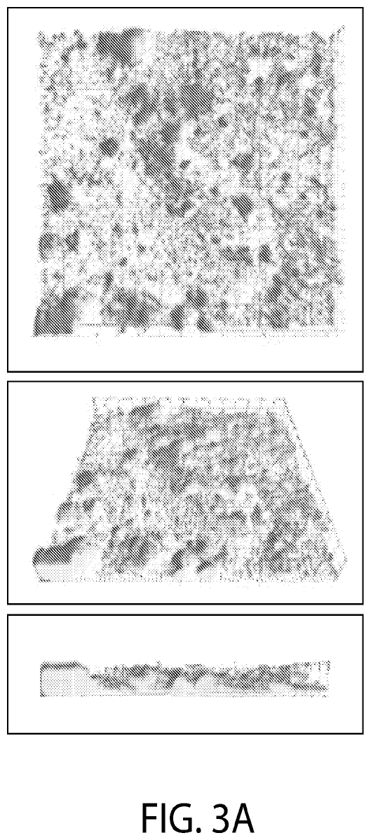

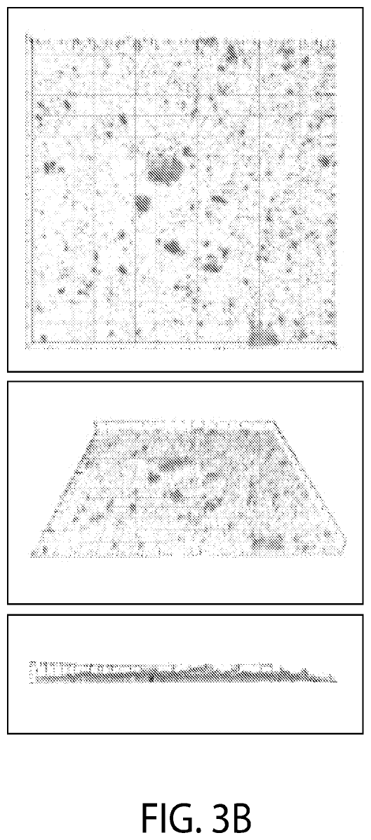

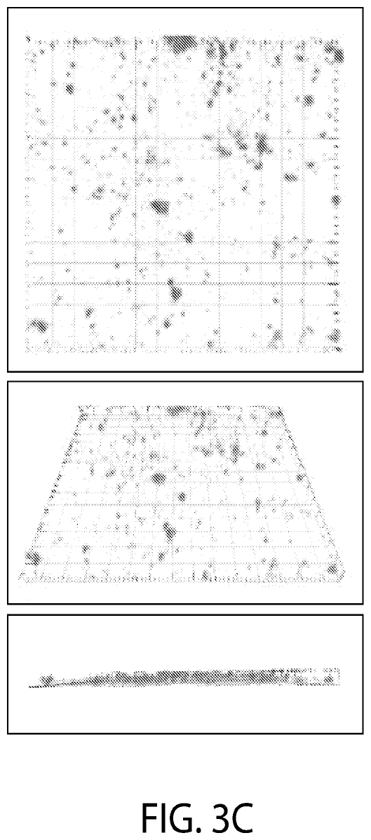

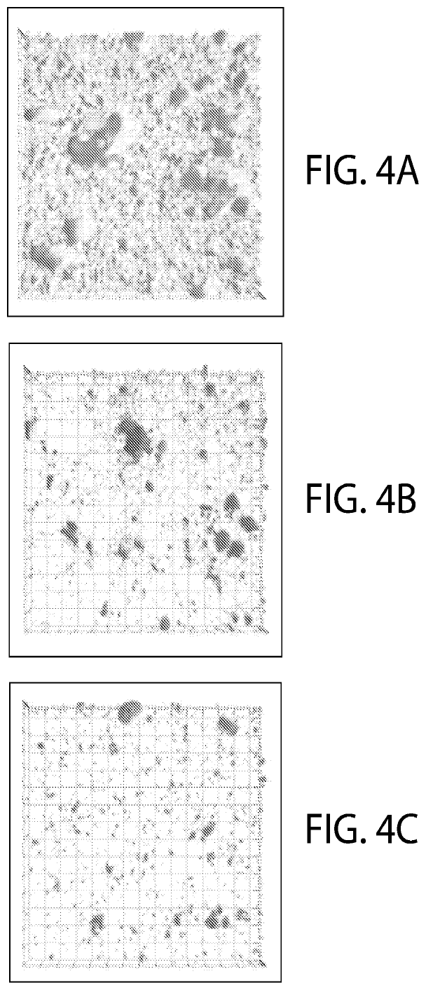

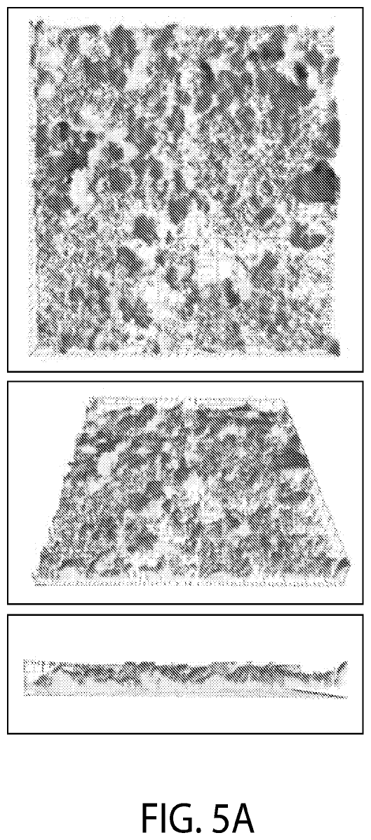

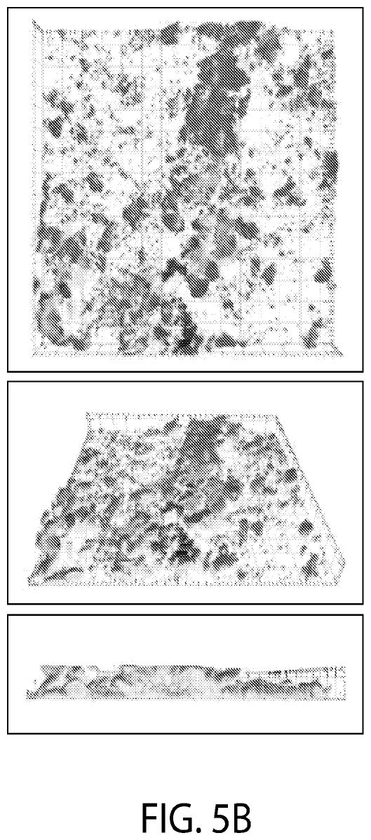

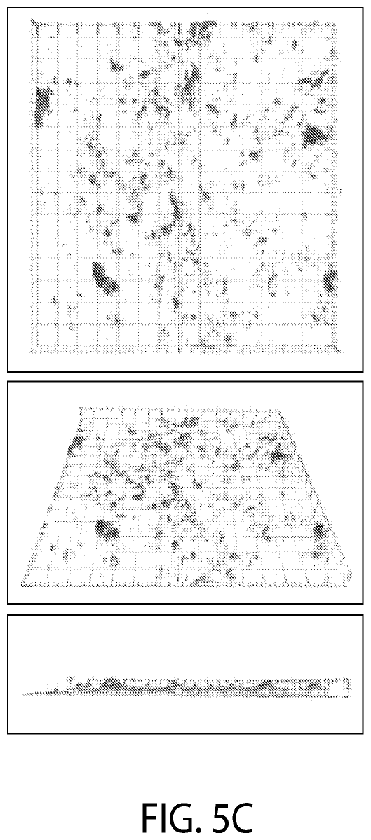

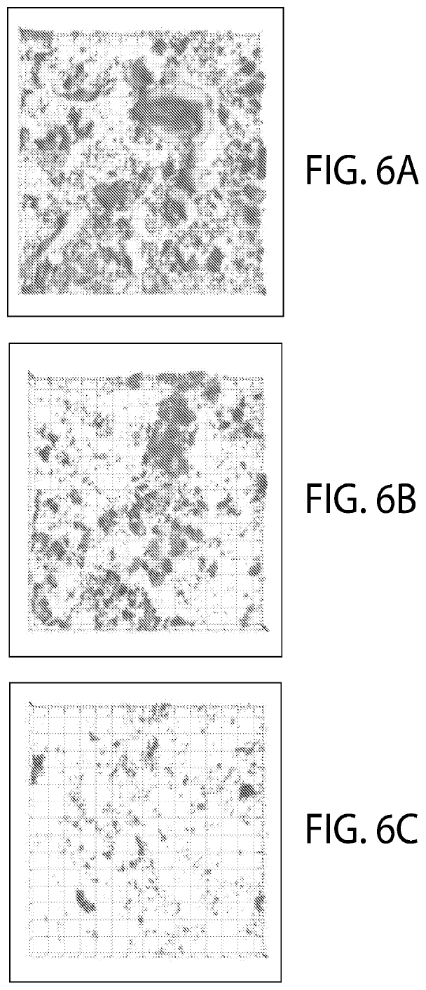

The impact on biofilm thickness and roughness provided by exemplary compositions of the present invention was evaluated in accordance with the methods described hereinabove in Example 1. The results of these experiments demonstrate that exemplary compositions of present invention provide an unexpectedly beneficial impact on the surface topography and thickness of biofilm (see, e.g., FIG. 1 and FIG. 2).

It will be understood that while the invention has been described in conjunction with specific embodiments thereof, the foregoing description and examples are intended to illustrate, but not limit the scope of the invention. Other aspects, advantages and modifications will be apparent to those skilled in the art to which the invention pertains, and these aspects and modifications are within the scope of the invention and described and claimed herein.

1. A method for modifying the surface topography of an oral biofilm comprising administering an oral care composition comprising an effective amount of a basic amino acid or a divalent metal ion source, to the oral cavity of a subject in need thereof, wherein the basic amino acid is selected from arginine, lysine, histidine and a combination of two or more thereof, and wherein the divalent metal ion source is selected from a zinc ion source, a stannous ion source, and a combination of two or more thereof.

2-4. (canceled)

5. The method according to claim 1, wherein the arginine comprises L-arginine.

6. (canceled)

7. The method according to claim 1, wherein the effective amount of basic amino acid or divalent metal ion is an amount required to increase the roughness of a portion of a biofilm.

8. The method according to claim 7, wherein the roughness of a portion of the biofilm is increased by at least about 50%.

9. The method according to claim 7, wherein the roughness is measured using an apparatus comprising:
a frame having a first side, a second side, and an aperture extending from the first side to the second side;
a first cover attached to the first side and covering the aperture;
a second cover attached to the second side and covering the aperture, a well formed by the frame, the first cover, and the second cover;
a first tube fluidly coupled to the well; and
a second tube fluidly coupled to the well.

10. The method according to claim 7, wherein the roughness is measured with a skidded gage.

11. (canceled)

12. The method according to claim 7, wherein the roughness is measured using confocal microscopy.

13. The method according to claim 1, wherein the oral care composition is administered to the oral cavity for from about 10 seconds to about 10 minutes.

14. The method according to claim 1, wherein the oral care composition is administered to the oral cavity for from about 30 seconds to about 2 minutes.

15. The method according to claim 1, wherein the basic amino acid or the divalent metal ion source is present in an amount of from about 0.1% to about 10%, by weight of the oral care composition.

16-18. (canceled)

19. The method according to claim 1, wherein the divalent metal ion source comprises a zinc ion source selected from: zinc oxide; zinc citrate; zinc lactate; zinc phosphate; zinc chloride; and a combination of two or more thereof.

20-25. (canceled)

26. The method according to claim 1 further comprising:
dislodging at least a portion of the biofilm from an oral cavity surface.

27. The method according to claim 1, wherein the method further comprises the step of expectorating at least a portion of the oral care composition.

28. (canceled)

29. A method for reducing the thickness of an oral biofilm comprising administering an oral care composition to the oral cavity of a subject in need thereof, the oral care composition comprising:
an effective amount of a basic amino acid selected from arginine, lysine, histidine, and a combination of two or more thereof;
a divalent metal ion source selected from a zinc ion source, a stannous ion source, and a combination of two or more thereof;
an abrasive;
a flavorant; and
a thickening agent.

30-33. (canceled)

34. The method according to claim 29, wherein the thickness of a portion of the biofilm is decreased by at least about 25%.

35. The method according to claim 29, wherein the thickness is measured using an apparatus comprising:
a frame having a first side, a second side, and an aperture extending from the first side to the second side;
a first cover attached to the first side and covering the aperture;
a second cover attached to the second side and covering the aperture, a well formed by the frame, the first cover, and the second cover;
a first tube fluidly coupled to the well; and
a second tube fluidly coupled to the well.

36-37. (canceled)

38. The method according to claim 29, wherein the basic amino acid or the divalent metal ion source is present in an amount of from about 0.1% to about 10%, by weight of the oral care composition.

39-45. (canceled)

46. The method according to claim 29, wherein reducing the thickness of the biofilm makes the biofilm susceptible to penetration by an ingredient.

47-66. (canceled)

67. The method according to claim 29, wherein the biofilm is a single species or a mixed-species biofilm, the biofilm being present on an inanimate surface.

68-82. (canceled)

83. The method according to claim 29, wherein the method further provides inhibition of biofilm growth.
